SEQUENCE LISTING (14 sequences)

SEQ ID NO: 1 - DNA, 5040 bp, Escherichia coli
  promoter (71)..(99): factor Sigma 70; predicted +1 start at 106
agcttttcattctgactgcaacgggcaatatgtctctgtgtggattaaaaaaagagtgtctgatagcagcttctgaactggttacctgccgtgagtaaattaaaattttattgacttaggtcactaaatactttaaccaatataggcatagcgcacagacagataaaaattacagagtacacaacatccatgaaacgcattagcaccaccattaccaccaccatcaccattaccacaggtaacggtgcgggctgacgcgtacaggaaacacagaaaaaagcccgcacctgacagtgcgggctttttttttcgaccaaaggtaacgaggtaacaaccatgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctgacatggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaaatgaaactctacaatctgaaagatcacaacgagcaggtcagctttgcgcaagccgtaacccaggggttgggcaaaaatcaggggctgttttttccgcacgacctgccggaattcagcctgactgaaattgatgagatgctgaagctggattttgtcacccgcagtgcgaagatcctctcggcgtttattggtgatgaaatcccacaggaaatcctggaagagcgcgtgcgcgcggcgtttgccttcccggctccggtcgccaatgttgaaagcgatgtcggttgtctggaattgttccacgggccaacgctggcatttaaagatttcggcggtcgctttatggcacaaatgctgacccatattgcgggtgataagccagtgaccattctgaccgcgacctccggtgataccggagcggcagtggctcatgctttctacggtttaccgaatgtgaaagtggttatcctctatccacgaggcaaaatcagtccactgcaagaaaaactgttctgtacattgggcggcaatatcgaaactgttgccatcgacggcgatttcgatgcctgtcaggcgctggtgaagcaggcgtttgatgatgaagaactgaaagtggcgctagggttaaactcggctaactcgattaacatcagccgtttgctggcgcagatttgctactactttgaagctgttgcgcagctgccgcaggagacgcgcaaccagctggttgtctcggtgccaagcggaaacttcggcgatttgacggcgggtctgctggcgaagtcactcggtctgccggtgaaacgttttattgctgcgaccaacgtgaacgataccgtgccacgtttcctgcacgacggtcagtggtcacccaaagcgactcaggcgacgttatccaacgcgatggacgtgagtcagccgaacaactggccgcgtgtggaagagttgttccgccgcaaaatctggcaactgaaagagctgggttatgcagccgtggatgatgaaaccacgcaacagacaatgcgtgagttaaaagaactgggctacacttcggagccgcacgctgccgtagcttatcgtgcgctgcgtgatcagttgaatccaggcgaatatggcttgttcctcggcaccgcgcatccggcgaaatttaaagagagcgtggaagcgattctcggtgaaacgttggatctgccaaaagagctggcagaacgtgctgatttacccttgctttcacataatctgcccgccgattttgctgcgttgcgtaaattgatgatgaatcatcagtaaaatctattcattatctcaat

SEQ ID NO: 2 - protein, 21 aa, Escherichia coli [sequence omitted]

SEQ ID NO: 3 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 4 - protein, 310 aa, Escherichia coli
MVKVYAPASSANMSVGFDVLGAAVTPVDGALLGDVVTVEAAETFSLNNLGRFADKLPSEPRENIVYQCWERFCQELGKQIPVAMTLEKNMPIGSGLGSSACSVVAALMAMNEHCGKPLNDTRLLALMGELEGRISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 5 - protein, 428 aa, Escherichia coli
MKLYNLKDHNEQVSFAQAVTQGLGKNQGLFFPHDLPEFSLTEIDEMLKLDFVTRSAKILSAFIGDEIPQEILEERVRAAFAFPAPVANVESDVGCLELFHGPTLAFKDFGGRFMAQMLTHIAGDKPVTILTATSGDTGAAVAHAFYGLPNVKVVILYPRGKISPLQEKLFCTLGGNIETVAIDGDFDACQALVKQAFDDEELKVALGLNSANSINISRLLAQICYYFEAVAQLPQETRNQLVVSVPSGNFGDLTAGLLAKSLGLPVKRFIAATNVNDTVPRFLHDGQWSPKATQATLSNAMDVSQPNNWPRVEELFRRKIWQLKELGYAAVDDETTQQTMRELKELGYTSEPHAAVAYRALRDQLNPGEYGLFLGTAHPAKFKESVEAILGETLDLPKELAERADLPLLSHNLPADFAALRKLMMNHQ

SEQ ID NO: 6 - DNA, 66 bp, Escherichia coli
  (1)..(66): leader sequence
atgaaacgcattagcaccaccattaccaccaccatcaccattaccacaggtaacggtgcgggctga

SEQ ID NO: 7 - DNA, 34 bp, Escherichia coli
  (1)..(34): attenuator
aaaaaagcccgcacctgacagtgcgggctttttt

SEQ ID NO: 8 - DNA, 12 bp, Artificial Sequence
  Description of Artificial Sequence  XbaI linker for linkage to thrA and attenuater
gactctagagtc

SEQ ID NO: 9 - DNA, 22 bp, Artificial Sequence
  Description of Artificial Sequence  primer for amplifying Escherichia coli leader sequence in thr operon
tggttacctgccgtgagtaaat

SEQ ID NO: 10 - DNA, 26 bp, Artificial Sequence
  Description of Artificial Sequence  primer for amplifying Escherichia coli leader sequence in thr operon
atgttgtgtactctgtaatttttatc

SEQ ID NO: 11 - DNA, 25 bp, Artificial Sequence
  Description of Artificial Sequence  primer for amplifying Escherichia coli leader sequence in thr operon
ctctgtaatttttatctgtctgtgc

SEQ ID NO: 12 - DNA, 23 bp, Artificial Sequence
  Description of Artificial Sequence  primer for amplifying Escherichia coli leader sequence in thr operon
tttatctgtctgtgcgctatgcc

SEQ ID NO: 13 - DNA, 21 bp, Artificial Sequence
  Description of Artificial Sequence  primer for amplifying Escherichia coli leader sequence in thr operon
tgtgcgctatgcctatattgg

SEQ ID NO: 14 - DNA, 15 bp, Artificial Sequence
  Description of Artificial Sequence  primer for amplifying Escherichia coli leader sequence in thr operon
gcctatattggttaa

(19) United States
(12) Patent Application Publication
(10) Pub. No.: US 2006/0216796 A1
(43) Pub. Date: Sep. 28, 2006

(54) METHOD FOR PRODUCING L-AMINO ACID BY FERMENTATION

(76) Inventors: Kenichi Hashiguchi, Kawasaki (JP); Yuta Nakai, Kawasaki (JP); Hisao Itou, Kawasaki (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/382,766

(22) Filed: May 11, 2006

Related U.S. Application Data
(63) Continuation of application No. PCT/JP04/17536, filed on Nov. 18, 2004.

(30) Foreign Application Priority Data
Nov. 21, 2003 (JP) 2003-391826

(51) Int. Cl. C12P 13/04; C12N 15/74; C12N 1/21

(52) U.S. Cl. 435/106; 435/488; 435/252.33

(57) ABSTRACT

L-threonine or L-isoleucine is produced by culturing a bacterium which belongs to the genus Escherichia and has an ability to produce L-threonine or L-isoleucine, and wherein expression of a threonine operon is directed by its native promoter, and from which at least a leader sequence and an attenuator are deleted, in a medium and collecting the L-threonine or L-isoleucine from the medium.

This application claims priority under 35 U.S.C. §119 to Japanese Application Serial No. 2003-391826, filed Nov. 21, 2003, and is a continuation under 35 U.S.C. §120 of PCT Application No. PCT/JP2004/017536, filed on Nov. 18, 2004. The Sequence Listing on Compact Disk filed herewith is also hereby incorporated by reference in its entirety (File Name: US-185 Seq List; File Size: 39 KB; Date Created: May 11, 2006).

### BACKGROUND OF THE INVENTION

1. Technical Field

The present invention relates to a method for producing an L-amino acid using a bacterium belonging to the genus Escherichia. Specifically, the present invention relates to a method for producing L-threonine or L-isoleucine. L-threonine and L-isoleucine are both essential amino acids, and L-threonine is used as a component of various nutritional formulations for medical uses, or as a component in animal feed. L-isoleucine is not only useful as a drug, such as in nutrient preparations, but also as a feed additive.

2. Background Art

L-amino acids such as L-threonine and L-isoleucine are industrially produced by fermentation using amino acid-producing bacteria such as coryneform bacteria and bacteria belonging to the genus Escherichia, wherein said bacteria have the ability to produce these L-amino acids. L-amino acid-producing bacteria including strains separated from nature or artificially mutated strains thereof, recombinant strains which have an enhanced activity of an L-amino acid biosynthetic enzyme, and so forth, are used to improve the production of these L-amino acids.

Methods for producing L-threonine utilizing a mutant strain of Escherichia bacterium have been reported, and include a method of utilizing a 6-dimethylaminopurine-resistant strain (Japanese Patent Laid-open (Kokai) No. 5-304969), and a method of utilizing a borrelidin-resistant strain (International Patent Publication WO98/04715). Methods for producing L-threonine utilizing a recombinant strain of Escherichia bacterium have also been reported, and include a method of utilizing a strain in which the threonine operon is amplified with a plasmid (Japanese Patent Laid-open No. 05-227977), and a method of utilizing a strain in which the phosphoenolpyruvate carboxylase gene or the aspartase gene is amplified with a plasmid (U.S. Patent Application Laid-open No. 2002/0110876).

Methods for producing L-isoleucine utilizing a mutant strain of Escherichia bacterium have been reported, and include a method utilizing a 6-dimethylaminopurine-resistant strain (Japanese Patent Laid-open No. 5-304969), a method utilizing an L-isoleucine hydroxamate-resistant strain (Japanese Patent Laid-open No. 5-130882), and a method utilizing a thiaisoleucine-resistant strain (Japanese Patent Laid-open No. 5-130882). Methods for producing L-isoleucine utilizing a recombinant Escherichia bacterium have been reported, and include a method of using a strain in which the threonine deaminase gene or the threonine acetohydroxy acid synthase gene is amplified with a plasmid (Japanese Patent Laid-open No. 2-458, European Patent No. 0593729).

A method for producing L-threonine or L-isoleucine using a bacterium belonging to the genus Escherichia has been reported in which the expression of a gene coding for an enzyme involved in the biosynthesis of L-threonine or L-isoleucine is amplified.

Genes coding for enzymes involved in the biosynthesis of L-threonine in Escherichia coli have been reported, and include the aspartokinase III gene (lysC), the aspartate semialdehyde dehydrogenase gene (asd), the aspartokinase 1-homoserine dehydrogenase gene (thrA), the homoserine kinase gene (thrB), the threonine synthase gene (thrC), and so forth.

The thrABC sequence, a part of the threonine-biosynthetic pathway of Escherichia coli, forms the threonine operon.

Expression of the threonine operon is regulated by a decrease in the transcription by the intracellular concentrations of L-threonine and L-isoleucine, which is referred to as “attenuation.” Moreover, it has been reported that, inter alia, expression of the threonine operon in Escherichia coli is regulated via a regulatory sequence located between the threonine promoter and thrA. thrA is a structural gene of a threonine operon (Lynn S. P. et al., “Journal of Molecular Biology (J. Mol. Biol)”, Academic Press, vol. 183 (1985) pp. 529-541). Furthermore, it has also been reported that this regulatory sequence contains a leader sequence comprising several tens of nucleotides, as well as an attenuator, both of which are located between the promoter region and the initiation codon.

Many threonine and isoleucine codons are included in the leader sequence, and when threonine or isoleucine exists in the medium, translation of the leader sequence proceeds smoothly. As a result, the attenuator forms a three-dimensional structure, thereby decreasing transcription, and thus decreasing the expression of the threonine biosynthetic pathway genes. When threonine and isoleucine do not exist in the medium, movement of the ribosome on the leader sequence is slowed, and the expression of the threonine biosynthetic pathway genes increases due to the change in the three-dimensional structure of the mRNA.

The efficient production of L-threonine in the presence of high concentrations of isoleucine and threonine has been attempted by releasing the attenuation to allow high expression of the threonine operon.

It has been reported that the threonine operon is slightly regulated by the attenuation, and its expression increases when a threonine operon lacking the attenuator is ligated with a potent heterogenous promoter that allows high expression of the operon. It has also been reported that a bacterium containing this threonine operon has increased L-threonine-producing ability (Japanese Patent Laid-open No. 05-227977). Furthermore, it has been disclosed that conferring borrelidin-resistance to a bacterium changes the threoninyl-tRNA synthase activity, and thereby the threonine operon comes to be slightly regulated by the attenuation. Thus, the L-threonine-producing ability can be improved (International Patent Publication WO98/04715).

However, when only the attenuator is removed, reduction of transcription occurs by addition of L-isoleucine or L-threonine to the medium, and the expression of the threonine operon is still insufficient despite release of the attenuation. Therefore, in the fermentation of L-threonine and L-isoleucine having increased concentrations of L-threonine and L-isoleucine in the medium, a further increase in the expression of the threonine operon is desirable. Conversely, when a heterologous promoter is used to direct the expression of the threonine operon, expression is significantly affected by such factors as the distance between the promoter and the transcription initiation site, the distance between the SD sequence and the initiation codon, and the sequence of the initiation codon. Therefore, it is difficult to obtain a maximum and stable expression. Thus, the creation of a strain having a stable L-isoleucine or L-threonine-producing ability using the native promoter has long been desirable (Dalboge H. et al., “DNA”, New York Ny Mary Ann Liebert, July and August, 1988, Vol. 7, No. 6, pp. 399-405).

### SUMMARY OF THE INVENTION

An object of the present invention is to improve the ability of a bacterium belonging to the genus Escherichia to produce an L-amino acid, especially, L-threonine and L-isoleucine, by enhancing the threonine biosynthetic pathway in the bacterium.

The inventors of the present invention assiduously studied in order to achieve the aforementioned object, and as a result, they succeeded in constructing a threonine operon that is not subject to regulation by attenuation mediated by isoleucine and threonine in a medium. This was accomplished by removing at least the leader sequence and the attenuator in the attenuation region. They also found that a strain having such a threonine operon exhibited superior properties in the production of L-threonine or L-isoleucine by fermentation, and thus accomplished the present invention.

It is an object of the present invention to provide a bacterium belonging to the genus Escherichia which has an ability to produce L-threonine or L-isoleucine, wherein expression of a threonine operon therein is directed by a native promoter, and wherein at least a leader sequence and an attenuator has been deleted from said operon.

A further object of the present invention is to provide the Escherichia bacterium as described above, wherein said threonine operon is on a plasmid.

It is a further object of the present invention to provide the Escherichia bacterium as described above, wherein said threonine operon is on a chromosome.

It is a further object of the present invention to provide the Escherichia bacterium as described above which has an ability to produce L-isoleucine, and wherein an activity of an L-isoleucine biosynthetic enzyme is enhanced.

It is a further object of the present invention to provide a threonine operon comprising a native promoter and thrABC, wherein at least the leader sequence and the attenuator are deleted therefrom.

It is a further object of the present invention to provide the threonine operon as described above comprising the sequence shown in SEQ ID NO: 1, wherein at least the sequence of nucleotides 188 to 310 has been deleted.

It is a further object of the present invention to provide the threonine operon as described above comprising the sequence shown in SEQ ID NO: 1, wherein at least the sequence of nucleotides 168 to 310 has been deleted.

It is a further object of the present invention to provide the threonine operon as described above, comprising the sequence shown in SEQ ID NO: 1, wherein at least the sequence of nucleotides 148 to 310 has been deleted.

It is a further object of the present invention to provide a method for producing L-threonine or L-isoleucine comprising culturing the bacterium as described above in a medium, and collecting the L-threonine or L-isoleucine from the medium.

### BRIEF DESCRIPTION OF THE DRAWINGS

FIG. 1 shows a scheme for constructing a plasmid which is used for amplifying the threonine operon which lacks the attenuator.

FIG. 2 shows a scheme for constructing a plasmid which is used for amplifying the threonine operon which lacks the region involved in attenuation.

FIG. 3 shows a scheme for constructing a temperature-sensitive plasmid which is used for introducing into a chromosome a threonine operon which lacks the region involved in attenuation.

### DETAILED DESCRIPTION OF THE PREFERRED EMBODIMENTS

Hereinafter, the present invention will be explained in detail.

<1> Bacterium of the Present Invention

The bacterium of the present invention is a bacterium which belongs to the genus Escherichia, has an ability to produce L-threonine or L-isoleucine, and has a modified threonine operon, whereby expression of the threonine operon is regulated by its native promoter. The modified threonine operon has a deleted region that includes at least a leader sequence and an attenuator, which results in prevention of the attenuation. Hereafter, this threonine operon is referred to as the “threonine operon of the present invention”. The bacterium of the present invention may have both L-threonine and L-isoleucine-producing abilities.

The bacterium of the present invention can be obtained either by introducing the threonine operon of the present invention into a bacterium belonging to the genus Escherichia and which has L-threonine or L-isoleucine producing-ability, or by imparting L-threonine or L-isoleucine-producing ability to a bacterium having the threonine operon of the present invention. In addition, the bacterium of the present invention may also be a bacterium that has L-threonine or L-isoleucine-producing ability because it has been modified to have the threonine operon of the present invention.

Although the parent strain of the bacterium belonging to the genus Escherichia used for obtaining the bacterium of the present invention is not particularly limited, those described in Neidhardt et al. (Neidhardt, F. C. et al., Escherichia coli and Salmonella Typhimurium, American Society for Microbiology, Washington D.C., 1029, Table 1) may be used. Those include, for example, Escherichia coli. Specific examples of Escherichia coli include Escherichia coli W3110 strain (ATCC 27325) derived from the K12 strain, which is a prototype wild-type strain, and Escherichia coli MG1655 (ATCC 47076).

These strains are available from the American Type Culture Collection (Address: 12301 Parklawn Drive, Rockville, Md. 20852, United States of America). Each strain is given a unique registration number which is listed in the catalogue of the American Type Culture Collection. Strains can be ordered by using this registration number.

<1>-1. Imparting L-Threonine or L-Isoleucine-Producing Ability

Hereinafter, a method for imparting L-threonine or L-isoleucine-producing ability to a bacterium belonging to the genus Escherichia will be described. In the present invention, the term “L-threonine-producing ability (ability to produce L-threonine)” means an ability of the bacterium of the present invention to produce and cause accumulation of L-threonine in a medium when it is cultured in the medium. In the present invention, the term “L-isoleucine-producing ability (ability to produce L-isoleucine)” means an ability of the bacterium of the present invention to produce and cause accumulation of L-isoleucine in a medium when it is cultured in the medium.

In order to impart L-threonine or L-isoleucine-producing ability, methods conventionally used for breeding an L-threonine or L-isoleucine-producing bacterium belonging to the genus Escherichia or Coryneform bacterium can be used. For example, methods for obtaining an auxotrophic mutant strain, analogue-resistant strain, or metabolic regulation mutant strain having L-threonine or L-isoleucine-producing ability, methods for creating a recombinant strain in which activity of an L-threonine-biosynthetic enzyme or an L-isoleucine biosynthetic-enzyme is enhanced, can be used. When breeding L-threonine or L-isoleucine-producing bacteria using these methods, other properties, such as auxotrophy, resistance to various analogues, and introduction of mutations which effect metabolic regulation, may also be imparted.

When a recombinant strain is created, activity of single or multiple L-threonine or L-isoleucine-biosynthetic enzymes may be enhanced. Furthermore, methods for imparting auxotrophy, resistance to various analogues, and introduction of mutations which effect metabolic regulation, may be combined with methods for enhancing an activity of L-threonine or L-isoleucine-biosynthetic enzyme.

A method for imparting L-threonine or L-isoleucine-producing ability to a bacterium belonging to the genus Escherichia by enhancing an activity of an L-threonine or L-isoleucine biosynthetic enzyme will be exemplified below. Enhancing an activity of an enzyme can be attained by, for example, introducing a mutation into a gene coding for the enzyme so that the intracellular activity of the enzyme is increased, or by utilizing a genetic recombination technique.

The genes encoding the L-threonine biosynthetic enzymes include aspartokinase III gene (lys), aspartate semialdehyde dehydrogenase gene (asd), and so forth. Names of genes coding for the respective enzymes are shown in the parentheses after the names of the enzymes. Two or more kinds of these genes may be introduced into a bacterium belonging to the genus Escherichia. These genes encoding the L-threonine biosynthetic enzymes may be introduced into a bacterium belonging to the genus Escherichia in which the threonine-degradation pathway is suppressed. Examples of bacterium in which the threonine-degradation pathway is suppressed include the TDH6 strain, which is deficient in threonine dehydrogenase activity (Japanese Patent Laid-open No. 2001-346578).

Examples of genes encoding the L-isoleucine-biosynthetic enzymes include threonine deaminase gene (ilvA), ketol-acid reductoisomerase gene (ilvC), acetolactate synthase gene (ilvI), dihydroxy-acid dehydratase gene (dad), and aminotransferase gene (ilvE). Names of genes coding for their respective enzymes are shown in the parentheses after the names of the enzymes. Two or more kinds of these genes may be introduced. The aforementioned ilvA and ilvE genes are contained in the ilvGMEDA operon (Japanese Patent Laid-open No. 2002-051787), and thus they may be introduced in the form of the ilvGMEDA operon.

Furthermore, L-threonine is a precurser to L-isoleucine. Therefore, in order to increase the L-isoleucine producing-ability, it is preferable to increase the supply of L-threonine. Thus, increasing the L-isoleucine-producing ability can be obtained by enhancing both the L-threonine biosynthetic pathway and the L-isoleucine biosynthetic pathway, as well as solely enhancing the biosynthetic pathway to L-isoleucine. Examples of bacteria imparted with L-threonine-producing ability in such a manner include those described in Japanese Patent Laid-open Nos. 2002-51787 and 9-121872.

Activities of any of the enzymes encoded by the aforementioned genes can be enhanced by, for example, amplifying the gene using a plasmid which is autonomously replicable in bacteria belonging to the genus Escherichia. Furthermore, the gene encoding the biosynthetic enzyme may also be introduced into the chromosome. Furthermore, the activities can also be enhanced by introducing into a bacterium a gene containing a mutation that results in enhancing the intracellular activity of the enzyme encoded by the gene. Examples of such mutations include a promoter sequence mutation that increases the transcription amount of the gene, and a coding region mutation that increases the specific activity of an enzyme encoded by the gene.

Gene expression can also be enhanced by replacing an expression regulatory sequence, such as a promoter, on a chromosomal DNA or plasmid with stronger one (International Patent Publication WO00/18935). Examples of such promoters include, but are not limited to, lac promoter, trp promoter, frc promoter and P_{R} promoter derived from lambda phage, and so forth. Methods for modifying the promoter may be combined with methods for increasing the copy number of a gene.

Specific examples of bacteria belonging to the genus Escherichia which are imparted with L-threonine or L-isoleucine-producing ability and can be used in the present invention will be exemplified below. However, the bacteria are not limited to the examples, but encompass any bacteria which have L-threonine or L-isoleucine-producing ability.

Examples of the bacteria imparted with L-threonine-producing ability include the 6-dimethylaminopurine-resistant strain (Japanese Patent Laid-open No. 5-304969), a strain in which a mutated gene of threonine-biosynthetic enzyme which causes overproduction of the enzyme is amplified with a plasmid (Japanese Patent Publication (Kokoku) No. 1-29559 and Japanese Patent Laid-open Nos. 5-2227977), and a strain in which a gene coding for pyruvate carboxylase and a gene coding for nicotinamide nucleotide transhydrogenase are both amplified (Japanese Patent Laid-open No. 2002-51787).

Furthermore, the Escherichia coli VKPM B-3996 (cf U.S. Pat. No. 5,175,107) may also be used. Escherichia coli VKPM B-3996 was deposited at the Russian National Collection of Industrial Microorganisms (VKPM GNII Genetika Address: Dorozhny proezd 1, Moscow 113545, Russia) on Apr. 7, 1987 with a registration number of VKPM B-3996. VKPM B-3996 strain harbors the plasmid pVIC40 (International Patent Publication WO90/04636) which is obtained by introducing a gene of threonine operon (thrABC) into a plasmid pAYC32 having a streptomycin-resistance marker gene (refer to Chistorerdov, A. Y., Tsygankov, Y. D., Plasmid, 1986, 16, 161-167). In pVIC40, the L-threonine-mediated feedback inhibition of the aspartokinase 1-homoserine dehydrogenase I encoded by thrA is released.

Examples of bacteria belonging to the genus Escherichia imparted with L-isoleucine-producing ability include the 6-dimethylaminopurine-resistant strain (Japanese Patent Laid-open No. 5-304969), the L-isoleucine hydroxamete-resistant strain (Japanese Patent Laid-open No. 5-130882), a thiaisoleucine-resistant strain (Japanese Patent Laid-open No. 5-130882), a DL-ethionine-resistant strain (Japanese Patent Laid-open No. 5-130882), an arginine hydroxamete-resistant strain (Japanese Patent Laid-open No. 5-130882), as well as strains in which a gene coding for threonine deaminase or acetohydroxy acid synthase, which is an L-isoleucine-biosynthesis enzyme, is amplified with a plasmid (Japanese Patent Laid-open Nos. 2-458, 2-42988, 8-47397).

<1>-2. Threonine Operon of the Present Invention

It is known that the transcription of the threonine operon is decreased by the transcriptional regulation called “attenuation” in the presence of high concentrations of isoleucine orthreonine (J. Mol. Biol. (1985) 183, 529-541). Release of this attenuation is important for increased production of these amino acids.

The threonine operon contains the thrABC structural genes, a native promoter upstream to the structural genes, and a region involved in attenuation which includes a leader sequence and a specific sequence called the “attenuator” which regulates the expression of the thrABC structural genes.

Examples of the leader sequence include, but are not limited to, the sequence shown in SEQ ID NO: 6. This sequence encodes a leader peptide consisting of 21 amino acid residues shown in SEQ ID NO: 2, and consists of a region coding for 8 threonine codons and 4 isoleucine codons and a region containing a termination codon.

Examples of the attenuator include, but are not limited to, the region having the sequence shown in SEQ ID NO: 7. This attenuator sequence contains two regions that are complementary to each other so that they can hybridize to each other, forming a three-dimensional structure (J. Mol. Biol. (1985) 183, 529-541). The attenuator, thereforeacts as a terminator, and terminates transcription. The hybridization of the complementary regions in the attenuator form a three-dimensional structure called “a stem loop structure,” and transcription is terminated at this point.

The reduction of transcription by attenuation in the presence of high concentrations of threonine and isoleucine occurs according to the following mechanism. When intracellular concentrations of isoleucine and threonine are high, concentrations of threoninyl-tRNA and isoleucyl-tRNA in the culture medium increase. Therefore, tRNA-amino acid complexes are present in the cells in an amount sufficient for translation of the leader sequence, which codes for many threonine and isoleucine codons. Thus, the leader sequence is smoothly transcribed and translated, and the translation is terminated at the termination codon of the leader sequence itself. Then, the complementary sequences within the attenuator hybridize to each other to form a stem loop structure, which terminates the transcription. Therefore, it is difficult for transcription to proceed up to the structural genes of threonine operon, and thus expression of the genes encoding the threonine biosynthetic enzymes decreases.

Conversely, when intracellular concentrations of threonine and isoleucine are low, the intracellular concentrations of threoninyl-tRNA and isoleucyl-tRNA decrease. Therefore, tRNA-amino acid complexes do not exist in an amount sufficient for translation of a leader sequence region, and thus a ribosome stops at a threonine codon or isoleucine codon in the leader sequence. As a result, the leader sequence is not translated smoothly, and a region immediately upstream of the termination codon in the coding region of the leader peptide and a region immediately upstream of the attenuator form a pair to inhibit the hybridization of complementary sequences within the attenuator. Thus, a terminator structure cannot be formed, and transcription is not terminated. Therefore, the transcription proceeds to the thrABC structural genes of the operon, resulting in maximal transcription of the structural genes of threonine operon and maximal production of the threonine biosynthetic enzymes.

When production of L-threonine and L-isoleucine is increased, intracellular concentrations of L-threonine and L-isoleucine are increased, and the regulation by attenuation functions to decrease the expression of the structural genes of the threonine operon. As a result, activities of threonine biosynthetic enzymes are reduced, and thus the ability to produce L-threonine or L-isoleucine cannot be exerted to the maximum extent.

If such regulation by attenuation could be released or prevented, the threonine operon would be expressed at a high level. In addition, release of attenuation can be combined with the enhancement of the L-threonine or L-isoleucine-producing ability as described above to further improve the ability to produce L-threonine or L-isoleucine.

The threonine operon encompasses “a promoter which is native to the threonine operon, and the thrABC structural genes, wherein at least a leader sequence and attenuator are deleted therefrom.”

The term “attenuation” indicates a reduction in transcription of the threonine operon structural genes due to an increase of intracellular concentrations of threonine and isoleucine. The “attenuator” indicates a region which has a sequence that can form a stem loop structure in the molecule, and which therefore acts to terminate transcription of the structural genes. Examples of such a sequence derived from a bacterium belonging to the genus Escherichia include the sequence shown in SEQ ID NO: 7. The “leader sequence” refers to a sequence that contains a high number of isoleucine codons and threonine codons, and examples of such a sequence derived from a bacterium belonging to the genus Escherichia include the sequence shown in SEQ ID NO: 6, which encodes the leader peptide containing 4 isoleucine residues and 8 threonine residues shown in SEQ ID NO: 2 (J. Mol. Biol. (1985) 183, 529-541). The “native promoter” refers to the promoter of the threonine operon itself, and examples of such a promoter derived from a bacterium belonging to the genus Escherichia include the promoter having a sequence of nucleotides 71 to 99, and/or a sequence of nucleotides 104 to 132 of SEQ ID NO: 1. Furthermore, the phrase “thrABC structural genes” means a polycistron containing the structural gene encoding aspartokinase 1-homoserine dehydrogenase (thrA), the structural gene encoding homoserine kinase (thrB), and the structural gene encoding threonine synthase (thrC). Examples of thrABC structural genes derived from a bacterium belonging to the genus Escherichia include a sequence of the nucleotides 337 to 5020 of SEQ ID NO: 1. The “thrABC structural genes” may be modified, so long as they encode proteins which have activities of aspartokinase 1-homoserine dehydrogenase, homoserine kinase, and threonine synthase. For example, like the thrABC gene contained in pVIC40 as described above, the thrABC structural genes may be modified so that the L-threonine-mediated feedback inhibition is eliminated.

The phrase “region involved in the attenuation” means a region which is located between the promoter and the thrA initiation codon, and contains at least a leader sequence and an attenuator. It is also referred to as an “attenuation region” and examples of such a region derived from a bacterium belonging to the genus Escherichia include a region having nucleotide numbers 148 to 310 in SEQ ID NO: 1 (J. Mol. Biol. (1985) 183, 529-541).

In the present invention, the phrase “regulation by attenuation is released” means that, due to removal of at least the leader sequence and attenuator from the threonine operon, the attenuator becomes unable to form a stem loop structure, and thus expression of the structural genes of the threonine operon in the presence of high concentrations of isoleucine or threonine is increased as compared with a wild-type strain or non-mutated strain.

Furthermore, the phrase “threonine operon in which at least the leader sequence and attenuator are deleted therefrom,” means that the threonine operon has a sequence that lacks at least the leader sequence and attenuator. So long as the attenuation is released, the sequence upstream to the leader sequence and/or the sequence between the leader sequence and the attenuator may also be deleted. For example, the leader sequence, attenuator, a sequence between the leader sequence and the attenuator, and a sequence on the 5′ side (upstream) of the leader sequence may be removed. Examples of the sequence between the leader sequence and attenuator include the sequence of the nucleotides 256 to 272 in the sequence of SEQ ID NO: 1, and so forth. Examples of the sequence on the 5′ side of the leader sequence include the sequence from the 168th to 189th nucleotides of SEQ ID NO: 1, the sequence from 148th to 189th nucleotides SEQ ID NO: 1, and so forth. As long as the attenuation is released, a sequence on the 5′ side of these sequences may be further removed.

A threonine operon obtained by modifying the threonine operon derived from a bacterium belonging to the genus Escherichia is preferred as the “threonine operon” of the present invention. Examples of the threonine operon of the present invention include the sequence of SEQ ID NO: 1 from which at least the sequences of SEQ ID NO: 6 and SEQ ID NO: 7 are deleted, and a homolog thereof. Specifically, the sequence of SEQ ID NO: 1, whereby at least the sequence of the nucleotide numbers 188 to 310 is deleted, is preferred; the sequence of SEQ ID NO: 1, whereby at least the sequence of the nucleotide numbers 168 to 310 is deleted, is more preferred; and the sequence of SEQ ID NO: 1, whereby at least the sequence of the nucleotide numbers 148 to 310 is deleted, is particularly preferred. The homolog of the threonine operon used in the present invention may be a threonine operon having a sequence which includes substitution, deletion, or insertion of one or several nucleotides from SEQ ID NO: 1, from which at least the sequences of SEQ ID NO: 6 and SEQ ID NO: 7 are deleted, so long as the threonine operon is not regulated by attenuation and expresses enzymatically-active thrA, B and C proteins. The term “several” as used herein is intended to mean 2 to 50, preferably 2 to 10, more preferably 2 to 5. Furthermore, the homolog of the threonine operon used in the present invention may also be a threonine operon which is hybridizable with a DNA having the nucleotide sequence of SEQ ID NO: 1, from which at least the sequences of SEQ ID NO: 6 and SEQ ID NO: 7 are deleted, under stringent conditions, so long as the threonine operon is not regulated by attenuation and expresses enzymatically-active ThrA, B and C proteins. Examples of the stringent conditions include, for example, washing one time, preferably two or three times, at salt concentrations of 1×SSC and 0.1% SDS, preferably 0.1×SSC and 0.1% SDS, at 60° C. after hybridization.

Furthermore, the aforementioned sequences may contain a sequence that cannot function as a leader sequence or attenuator at the site of the deleted region. Examples of the sequence that cannot function as a leader sequence or attenuator include a leader sequence in which all or a part of threonine codons or isoleucine codons are replaced with codons of other amino acids or a termination codon, an attenuator modified so that it cannot form a stem loop structure, and so forth.

In the present invention, the phrase “the expression of the structural genes of threonine operon increases” means that transcription of mRNA of the structural genes increases because of the release of the attenuation, and thereby the amount of translated thrABC protein increases. In the present invention, the phrase “specific activities of threonine biosynthetic enzymes encoded by the threonine operon increase” means that due to the increase in the expression of the structural genes of the threonine operon, specific activities of aspartokinase I-homoserine dehydrogenase (thrA), homoserine kinase (thrB) or threonine synthase (thrC) encoded by the structural genes, that is, the thrABC sequence, are increased as compared with that of a wild-type strain or parent strain. An example of the wild-type strain of Escherichia coli serving as the strain for comparison includes Escherichia coli W3110 (ATCC 27325), MG1655 (ATCC 47076).

A bacterium belonging to the genus Escherichia which contains the threonine operon of the present invention as described above can be obtained by preparing a DNA “from which at least the leader sequence and attenuator have been removed” by site-directed mutagenesis, or the like, and introducing the resulting DNA into the region involved in the attenuation of the chromosomal threonine operon, according to a method described herein. Furthermore, such a bacterium can also be obtained by amplifying a vector DNA carrying the threonine operon of the present invention in a bacterium belonging to the genus Escherichia. Examples of the vector DNA useful for this purpose include plasmids autonomously replicable in a bacterium belonging to the genus Escherichia, as described herein. Introduction of a mutation for deletion can be attained by, for example, using a commercially available genetic mutagenesis kit, restriction enzymes, PCR, and so forth, in combination.

The region involved in the attenuation of the threonine operon can also be modified by subjecting an Escherichia bacterium to a mutagenesis treatment such as ultraviolet irradiation, X-ray irradiation, radiation exposure, or treatment with a mutagenesis agent such as N-methyl-N′-nitrosoguanidine (NTG) or EMS (ethyl methanesulfonate), and selecting a bacterium in which the attenuation is released.

Increase of the expression of the threonine operon structural genes due to the release of attenuation in the bacterium of the present invention can be confirmed by measuring an enzymatic activity of one or more of the threonine biosynthetic enzymes encoded by the thrABC sequence in the bacterium which has been cultured in the presence of high concentrations of L-threonine or L-isoleucine. In this procedure, comparison is preferably made by measuring the enzymatic activity in the bacterium which have been cultured in L-threonine and L-isoleucine-depleted medium, since attenuation does not occur in this environment.

The enzymatic activity of homoserine dehydrogenase can be measured by the method described in Truffa-Bachi P., Le Bras G., Cohen G. N., Biochem. Biophys. Acta., 128:450 (1966), and enzymatic activities of homoserine kinase and threonine synthase can be measured by the method described in Parsot C., EMBO J. 1986 Nov., 5(11):3013-9. Furthermore, cellular proteins can be quantified with Protein Assay (Bio-Rad) using, for example, bovine serum albumin as a standard.

When the bacterium of the present invention is evaluated in terms of the homoserine dehydrogenase (hereinafter referred to as HD) activity, for example, preferred is the bacterium showing an HD activity of 25 nmol/min/mg of cellular protein or higher in the presence of high concentrations of threonine or isoleucine, the bacterium showing an HD activity of 2 to 3 times higher than a wild-type bacterium in the presence of high concentrations of threonine or isoleucine, or the bacterium which when cultured in the presence of high concentrations of threonine or isoleucine, exhibits HD activity not less than one third of the HD activity of the same bacterium cultured in the absence of threonine or isoleucine. However, the bacterium of the present invention is not limited to these. When the bacterium is cultured in the presence of high concentrations of threonine or isoleucine, L-isoleucine or L-threonine is preferably added at a concentration of 50 mg/L or higher.

As the DNA vector which can be used to introduce the threonine operon of the present invention into a bacterium belonging to the genus Escherichia, plasmid DNA is preferably used, and examples of plasmids for Escherichia coli include pSTV29 (Takara Bio), RSF1010 (Gene, vol. 75 (2), pp. 271-288, 1989), pUC19, pBR322, pMW119. In addition, phage DNA vectors may also be used. Examples of plasmids carrying the threonine operon of the present invention include a plasmid which is obtained by removing the region involved in attenuation from the plasmid pVIC40 (International Patent Publication in Japanese No. 3-501682), which carries the feedback inhibition-resistant type of threonine operon and is harbored by the L-threonine-producing microorganism VKPM B-3996.

Introduction of the threonine operon of the present invention into a chromosome of a bacterium can be attained by, for example, homologous recombination using a genetic recombination technique (Experiments in Molecular Genetics, Cold Spring Harbor Laboratory Press (1972); Matsuyama, S. and Mizushima, S., J. Bacteriol., 162, 1196 (1985)). For example, the introduction can be attained by replacing a region including the attenuation region of a wild-type threonine operon on a chromosome with the fragment having an attenuation-released type of sequence. The phrase “attenuation-released type of sequence” as used herein means a sequence from the region involved in the attenuation and from which at least the leader sequence and attenuator are deleted.

The mechanism of the homologous recombination is as follows. When a plasmid having a sequence showing homology to a chromosomal sequence is introduced into a cell, it causes recombination at the site of the homologous sequence at a certain frequency, and the introduced plasmid as a whole is incorporated into the chromosome. If recombination is further caused at the site of the homologous sequence, the plasmid is removed again from the chromosome. Then, at some site where the recombination is caused, the introduced gene may be incorporated into the chromosome and the original chromosomal gene may be excised from the chromosome with the plasmid. By choosing such a strain, a strain in which the wild-type attenuation region on a chromosome is replaced with a fragment having the attenuation-released type of sequence can be obtained.

Such a genetic recombination method based on the homologous recombination has been already established, and methods of using a linear DNA, temperature sensitive plasmid, and so forth can be used.

Examples of the temperature-sensitive plasmid that can function in a bacterium belonging to the genus Escherichia include pMAN997 (International Patent Publication WO99/03998), pMAN031 (Yasueda, H. et al., Appl. Microbiol. Biotechnol., 36, 211 (1991)), pHSG415, pHSG422 (Hashimoto, Gotoh, T. et al, 16, 227-235 (1981)), and so forth.

Substitution of the target gene can be confirmed by analyzing the genes on a chromosome with Southern blotting or PCR. Methods for preparation of genes, hybridization, PCR, preparation of plasmid DNA, digestion and ligation of DNA and transformation used in the present invention are described in Sambrook, J., Fritsch, E. F., Maniatis, T., Molecular Cloning, Cold Spring Harbor Laboratory Press, 1.21 (1989).

When the threonine operon of the present invention is introduced, the copy number of the threonine operon may be increased by introducing multiple operons into the chromosome. For example, the threonine operon of the present invention may be introduced into the chromosome using Mu phage (Japanese Patent Laid-open No. 2-109985), transposon (Berg, D. E. and Berg, C. M., Bio/Technol., 1-147), or the like.

<2> Method for Producing L-Threonine or L-Isoleucine

L-threonine or L-isoleucine can be produced by culturing a bacterium which belongs to the genus Escherichia and has an ability to produce L-threonine or L-isoleucine, in which the expression of the threonine operon structural genes is increased by removing the attenuation region or by introducing a mutation into the region as described above, in a medium to produce and cause accumulation of L-threonine or L-isoleucine in the medium, and collecting the L-threonine or L-isoleucine from the medium. L-threonine and L-isoleucine may be produced simultaneously.

L-threonine or L-isoleucine can be produced using the bacterium of the present invention in a conventional manner with a typical medium containing a carbon source, nitrogen source, inorganic salts, and other organic trace nutrients, if required. Either a synthetic medium and/or a natural medium may be used. Any carbon source and nitrogen source may be used in the medium so long as they can be utilized by the strain to be cultured.

As the carbon source, sugars such as glucose, glycerol, fructose, sucrose, maltose, mannose, galactose, starch hydrolysate and molasses can be used, and organic acids such as acetic acid and citric acid and alcohols such as ethanol can also be used singly or in combination.

Ammonia, ammonium salts such as ammonium sulfate, ammonium carbonate, ammonium chloride, ammonium phosphate and ammonium acetate, nitric acid salts and so forth can be used as the nitrogen source.

Amino acids, vitamins, aliphatic acids, nucleic acids, substances containing these, such as peptone, casamino acid and decomposed product of soybean protein, and so forth, can be used as the trace amount of organic nutrients. When an auxotrophic mutant strain requiring an amino acid or the like for growth is used, the required nutrient is preferably supplemented. In particular, a threonine-producing bacterium showing isoleucine-auxotrophy is desirably cultured with supplementation of isoleucine which is required for growth.

Phosphates, magnesium salts, calcium salts, iron salts, manganese salts, and so forth can be used as the trace amount of organic nutrients.

The culture is preferably carried out under aerobic conditions at 25° C. to 45° C., and at a pH of 5 to 9. When the pH value decreases during the culture, calcium carbonate may be added, or the medium may be neutralized with an alkaline substance such as ammonia gas. Under such conditions, a marked amount of L-threonine or L-isoleucine accumulates in the medium after culturing for, preferably, about 10 to 120 hours.

Collection of the accumulated L-threonine or L-isoleucine from the medium after the culture can be accomplished by any conventional collection method. For example, the amino acids can be collected by removal of cells from the medium by centrifugation and subsequent crystallization by concentration.

### EXAMPLES

The present invention will be more specifically explained with reference to the following non-limiting examples.

### Example 1

### Construction and Evaluation of a Strain Harboring a Plasmid for Amplification of Threonine Operon from which the Attenuator is Removed

<1> Preparation of a Plasmid for Removal of Attenuator

The plasmid pVIC40 which is autonomously replicable in Escherichia coli and carries the threonine operon (International Patent Publication in Japanese No. 3-501682) was digested with the restriction enzymes HindIII and BamHI to obtain a fragment of about 6 kbp containing the threonine operon. Then, pBR322 (purchased from Takara Bio) was digested with the restriction enzymes HindIII and BamHI, and the aforementioned fragment of about 6 kbp containing the threonine operon was inserted into the digested pBR322 to obtain pBRT3240A. This pBRT3240A was treated with MluI, and an adapter having the restriction enzyme XbaI recognition site, which was obtained by hybridizing the oligonucleotide shown in SEQ ID NO: 8 and a complementary strand thereof, was inserted into the MluI site of pBRT3240A to obtain a plasmid pBR3240A.

Then, a fragment containing both the threonine promoter and the thrA gene, which codes for homoserine dehydrogenase, was amplified by PCR using pVIC40 as a template. The obtained fragment was inserted into the HincI site of pHSG399 (purchased from Takara Bio) to obtain pHSGthrA.

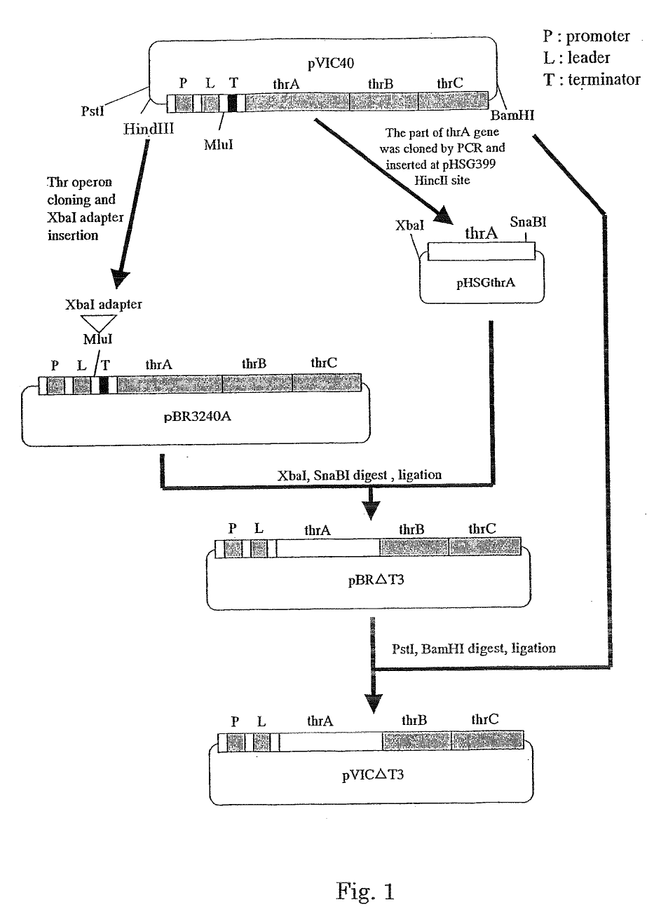

A fragment obtained by digesting pBRT3240A with the restriction enzymes XbaI and SnaBI and a fragment coding for the thrA region obtained by digesting pHSGthrA with XbaI and SnaBI were ligated to obtain a plasmid pBRAT3. Then, a fragment containing thrABC obtained by treating pBRAT3 with PstI and BamHI was introduced into a PstI- and BamHI-digested fragment of pVIC40 to obtain plasmid pVICΔT3. The plasmid pVICΔT3 is autonomously replicable in Escherichia coli and has a threonine operon including the region involved in the attenuation, from which only the attenuator is removed (FIG. 1).

Plasmid pVICΔT3, described above, and a control plasmid pVIC40, which has a wild-type attenuator, were used to transform the E. coli Gif33 strain which is deficient in homoserine dehydrogenase (AK-I, Theze J., Saint-Girons I., J. Bacteriol., 118(3):990 (1974)) according to the method of C. T. Chung (C. T. Chung, S. L. Niemela, R. H. Miller, Proc. Natl. Acad. Sci. (1989) vol. 86, pp. 2172-2175). A pVICΔT3-amplified transformant and a control wild-type threonine operon-amplified transformant were selected for streptomycin-resistance. The transformant obtained by introducing pVICΔT3 was designated Gif3/pVICΔT3, and the transformant obtained by introducing pVIC40 was designated Gif33/pVIC40.

Plasmids were extracted from the Gif3/pVICΔT3 and Gif33/pVIC40 strains selected as described above, and it was confirmed that the objective plasmids were respectively amplified in each strain.

<2> Culture of a Strain Harboring a Plasmid for Amplification of Threonine Operon from which the Attenuator is Removed and Measurement of Homoserine Dehydrogenase Activity

The transformant Gif33/pVICΔT3 in which the plasmid pVICΔT3 containing a threonine operon without the attenuator was amplified and the transformant Gif33/pVIC40 in which pVIC40 containing a wild-type attenuator was amplified were respectively cultured as described below, and homoserine dehydrogenase (henceforth referred to as HD) activities were measured in each strain.

Cells of Gif33/pVIC40 and Gif33/pVICΔT3 pre-cultured in the LB medium containing 20 μg/ml of streptomycin were respectively cultured in a production medium containing 40 g of glucose, 16 g of ammonium sulfate, 1 g of monopotassium phosphate, 0.01 g of ferrous sulfate heptahydrate, 0.01 g of manganese chloride tetrahydrate, 2 g of yeast extract, 1 g of magnesium sulfate heptahydrate, 50 mg or 250 mg of isoleucine and 30 g of calcium carbonate per 1 L of pure water (adjusted to pH 7.0 with KOH) at 37° C. for 22 to 27 hours with shaking at about 115 rpm.

After completion of the culture, the cells were collected from the medium, and the HD activity was measured according to the method described in Truffa-Bachi P., Le Bras G., Cohen G. N., Biochem. Biophys. Acta., 128:450 (1966), in which crude enzyme solution was added to the reaction mixture containing 200 mM Tris-HCl (pH 9.0), 500 mM KCl, 25 mM L-homoserine and 0.8 mM NADP, and the increase of absorbance at 340 nm was measured. As a control, the reaction solution containing water instead of homoserine was used. The crude enzyme solution was prepared by separating the cells from the aforementioned medium by centrifugation, washing the cells with 0.1 M KP buffer (0.01 M DTT, pH 7.0), then disrupting the cells by ultrasonication, and then removing undisrupted cells by centrifugation. Proteins in the crude enzyme solution were quantified with Protein Assay (Bio-Rad) using bovine serum albumin as a standard. The results are shown in Table 1.

TABLE 1

| | Added | HD activity |
|---|---|---|
| Strain | isoleucine (mg/L) | (nmol/mim/mg) |
| Gif33/pVIC40 | 50 | 11.6 |
| | 250 | 4.3 |
| Gif33/pVICΔT3 | 50 | 12.0 |
| | 250 | 4.5 |

As a result, no difference in the HD enzymatic activity was observed between the strains Gif33/pVICΔT and Gif33/pVIC40. This result demonstrates that expression of the threonine operon did not increase only as a result of removal of the attenuator.

### Example 2

### Construction and Evaluation of a Strain having a Threonine Operon from which a Different Segment of Sequence Including the Attenuator and Leader Sequence is Removed

<1> Construction of a Plasmid for Removal of the Attenuator and the Leader Sequence

As described above, the attenuation caused by addition of isoleucine could not be released as a result of the removal of the attenuator. Therefore, removal of not only the attenuator, but also the leader sequence, was attempted. First, PCR was performed by using pVIC40 as a template to obtain a fragment having a promoter and the subsequent region. PCR was performed by using the oligonucleotide shown in SEQ ID NO: 9, which is complementary to a sequence located in a region upstream to the promoter, and any of the oligonucleotides having the sequences of SEQ ID NOS: 10 to 14. Each of the obtained DNA fragments was purified in a conventional manner and ligated to pHSG398 (Takara Bio), which had been digested with HincII. Thereby, a plasmid pHPBthr which contains a fragment amplified with the oligonucleotides of SEQ ID NOS: 9 and 10, a plasmid pHPCthr which contains a fragment amplified with the oligonucleotides of SEQ ID NOS: 9 and 11, a plasmid pHPDthr which contains a fragment amplified with the oligonucleotides of SEQ ID NOS: 9 and 12, a plasmid pHPEthr which contains a fragmment amplified with the oligonucleotides of SEQ ID NOS: 9 and 13, and a plasmid pHPFthr which contains a fragment amplified with the oligonucleotides of SEQ ID NOS: 9 and 14 were obtained. Then, these five plasmids were digested with the restriction enzymes HindIII and BamHI, and the obtained fragments containing the upstream region of thrA were introduced into a HindIII-BamHI-digested pBR322 (Nippon Gene) to obtain plasmids pBRB, pBRC, pBRD, pBRE and pBRF.

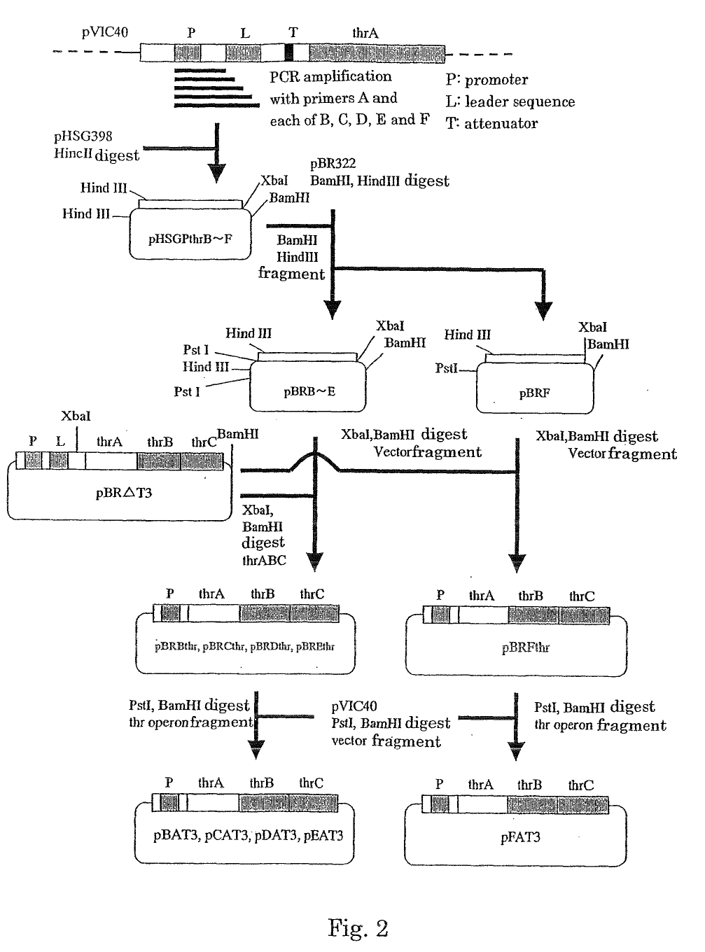

Then, the aforementioned plasmid pBRΔT3, which is for amplification of the threonine operon lacking the attenuator, was digested with XbaI and BamHI, and the obtained fragment containing thrABC was introduced into a XbaI-BamHI-digested pBRB, pBRC, pBRD, pBRE and pBRF to obtain plasmids pBRBthr, pBRCthr, pBRDthr, pBREthr and pBRFthr, each carrying the threonine operon from which a different segment of a sequence including the leader sequences and attenuator was removed. The fragments obtained by digesting plasmids pBRBthr, pBRCthr, pBRDthr, pBREthr and pBRFthr with PstI and BamHI were each introduced into the PstI-BamHI site of pVIC40, resulting in plasmids pBAT3, pCAT3, pDAT3, pEAT3 and pFAT3 (FIG. 2). The obtained plasmids pBAT3, pCAT3, pDAT3, pEAT3 and pFAT3 are autonomously replicable in Escherichia coli, and the attenuator and leader sequence of the attenuation region was completely removed, whereas a sequence upstream to the leader sequence was removed in different degrees. That is, the sequence having nucleotide numbers 188 to 310 of SEQ ID NO: 1 was removed in pBAT3, the sequence having nucleotide sequence numbers 178 to 310 was removed in pCAT3, the sequence having nucleotide sequence numbers 168 to 310 was removed in pDAT3, the sequence having nucleotide sequence numbers 158 to 310 was removed in pEAT3, and the sequence having nucleotide sequence numbers 148 to 310 was removed in pFAT3.

<2> Construction and Evaluation of the Strains Introduced with each Plasmid for Removal of Attenuation Region

For some of the obtained plasmids, effectiveness of the removal of the leader sequence and attenuator was tested. The plasmids pDAT3 and pFAT3 each lacking a different length of sequence including the leader sequence and attenuator and the control plasmid pVIC40 were respectively introduced into an HD-deficient Gif33 strain, and transformants were selected for streptomycin-resistance. The strains introduced with plasmids pDAT3 or pFAT3 were designated Gif33/pDAT3 or Gif33/pFAT3, respectively.

Plasmids were extracted from Gif33/pDAT3, Gif33/pFAT3, and the control Gif33/pVIC40 and it was confirmed that the objective plasmids were amplified in each strain. These transformants were cultured by the method described in <2> of Example 1, and the HD activity was measured. The results are shown in Table 2.

TABLE 2

| | Added | HD activity |
|---|---|---|
| Strain | isoleucine (mg/L) | (nmol/mim/mg) |
| Gif33/pVIC40 | 0 | 20.0 |
| | 250 | 3.4 |
| Gif33/pDAT3 | 0 | 17.7 |
| | 250 | 63.0 |
| Gif33/pFAT3 | 0 | 72.3 |
| | 250 | 46.2 |

The HD activity was decreased to about one sixth in the presence of isoleucine in the Gif33/pVIC40 strain, which contains an amplified threonine operon with a wild- and type attenuation region. In the Gif33/pFAT3 strains, which have the amplified threonine operon lacking the attenuator and leader sequence, the HD activity did not decrease in the presence of isoleucine strains. From the results of Example 1 and Example 2, strains Gif33/pDAT3 and Gif33/pFAT3 harboring a plasmid for amplification of threonine operon lacking the attenuator as well as leader sequence were not affected by attenuation caused by addition of isoleucine. The HD activity of the Gif33/pDAT3 strain was 17.7 nmol/min/mg in the absence of isoleucine, which was lower than the HD activity, 20.0 nmol/min/mg, of the control Gif33/pVIC40 strain. However, this is thought to be due to the curing of the plasmid during the culture.

Then, the TDH6 strain (Japanese Patent No. 3239903) obtained by curing pVIC40 from L-threonine-producing VKPMB-3996 was transformed with each of the plasmids pBAT3, pCAT3, pDAT3, pEAT3, pFAT3 which carry a threonine operon with attenuation-released sequence or with control plasmid pVIC40, and transformants were selected for streptomycin-resistance. The TDH6 strain had been modified so that it was deficient in threonine dehydrogenase activity by inserting transposon Tn5 (Japanese Patent Laid-open No. 2001-346578). The TDH6 strain is deposited at the Research Institute of Genetics and Selection of Industrial Microorganism (VNII Genetika, Address: Dorozhny proezd 1, Moscow 113545, Russia) on Aug. 15, 1987 with a registration number of VKPM B-3420.

The strains introduced with the plasmids pBAT3, pCAT3, pDAT3, pEAT3, pFAT3 or pVIC40 were designated TDH6/pBAT3 strain, TDH6/pCAT3 strain, TDH6/pDAT3 strain, TDH6/pEAT3 strain, TDH6/pFAT3 strain or TDH6/pVIC40 strain, respectively.

Plasmids were extracted from the transformants selected as described above and it was confirmed that the objective plasmids were amplified in each strain. These transformants were cultured by the method described in <2> of Example 1, and their L-threonine-producing abilities in the presence or absence of isoleucine were measured.

After completion of the culture, the amount of accumulated L-threonine in each culture broth was analyzed by liquid chromatography for appropriately diluted culture broth. The results are shown in Table 3. For each transformant, the amount of produced L-threonine are represented as relative values with respect to the amount of L-threonine produced in the absence of isoleucine, which was taken as 100.

TABLE 3

| | Added | Produced |
|---|---|---|
| | L-isoleucine | L-threonine as |
| Strain | (mg/L) | relative value |
| TDH6/pVIC40 | 0 | 100 |
| | 250 | 55 |
| TDH6/pBAT3 | 0 | 100 |
| | 250 | 60 |
| TDH6/pCAT3 | 0 | 100 |
| | 250 | 39 |
| TDH6/pDAT3 | 0 | 100 |
| | 250 | 76 |
| TDH6/pEAT3 | 0 | 100 |
| | 250 | 320 |
| TDH6/pFAT3 | 0 | 100 |
| | 250 | 79 |

Whereas the yield of threonine decreased to 55% in the presence of L-isoleucine compared to the yield obtained in the absence of isoleucine in the TDH6/pVIC40 strain, the yields obtained with the TDH6/pDAT3 strain, TDH6/pEAT3 strain and TDH6/pFAT3 strain in the presence of isoleucine in the medium were 76%, 320% and 79%, respectively. That is, the amount of L-threonine produced in the presence of isoleucine was slightly decreased, or even increased. Thus, it was demonstrated that with the sequence lacking the attenuator and leader sequence of the attenuation region, attenuation of the threonine operon did not occur, and the production of L-threonine was improved in the presence of high concentrations of L-isoleucine. As shown in Table 3 for the TDH6/pCAT3 strain, the amount of L-threonine produced in the presence of L-isoleucine was 39%, relative to the amount produced in the absence of isoleucine, which was lower than the value of the control strain TDH6/pVIC40. However, it is thought that this lower value was a result of the curing of the plasmid, and that the amount of L-threonine produced actually increased in this strain because of the elimination of the attenuation.

### Example 3

### Construction of a Strain in which Attenuator and Leader Sequence are Removed from its Chromosomal Threonine Operon and Evaluation of the Threonine Production of the Strain

<1> Construction of thrC Gene-Introduced TDH6 Strain

The attenuation-released type of sequence derived from the plasmid pDAT3 was introduced into a chromosome, and the effect thereof was determined. The TDH6 strain, an L-threonine-producing strain, lacks the thrC gene, which encodes threonine synthase. Therefore, TDH6 strain having a wild-type thrC was obtained by a conventional method using P1 transduction by using a Escherichia coli wild type W3110 strain (ATCC 27325) as a donor bacterium.

Specifically, this strain was obtained as follows. A culture of Escherichia coli W3110 strain and P1 phage dilution were added together to a soft agar medium maintained at a certain temperature, and the medium was spread over an LB plate. After the medium solidified, the cells were cultured at 37° C. for 6 to 7 hours to allow the phage to form plaques, and then phages were collected. The collected phages were added to the recipient TDH6 strain, and the cells were left standing at 37° C. for about 20 minutes in the presence of 2.5 mM CaCl₂ to allow adsorption of the phages, then reacted with 10 mM Na-citrate at 37° C. for about 30 minutes to terminate the adsorption reaction.

The TDH6 strain lacking thrC cannot grow in a minimal medium without threonine, whereas a strain introduced with thrC by P1 transduction can grow in the minimal medium. Then, the above reaction solution was inoculated into a minimal medium containing 0.5 g of glucose, 2 mM magnesium sulfate, 3 g of monopotassium phosphate, 0.5 g of sodium chloride, 1 g of ammonium chloride and 6 g of disodium phosphate per 1 L of pure water. A strain from a colony grown in the minimal medium after 24 hours was selected as a thrC-introduced strain and designated as W13.

<2> Construction of a Plasmid for Introducing a Threonine Operon having an Attenuation-Released Type of Sequence into a Chromosome

The plasmid pDAT3 carrying a threonine operon lacking the attenuator and leader sequence of the attenuation region (lacking the region having the nucleotide numbers 168 to 310 in SEQ ID NO: 1) was digested with HindIII and PvuII, and the obtained fragment containing the promoter, truncated attenuation region, and thrA was introduced into a HindIII-HincII-digested plasmid pUC18 (purchased from Takara Bio) to construct a plasmid pUC18D.

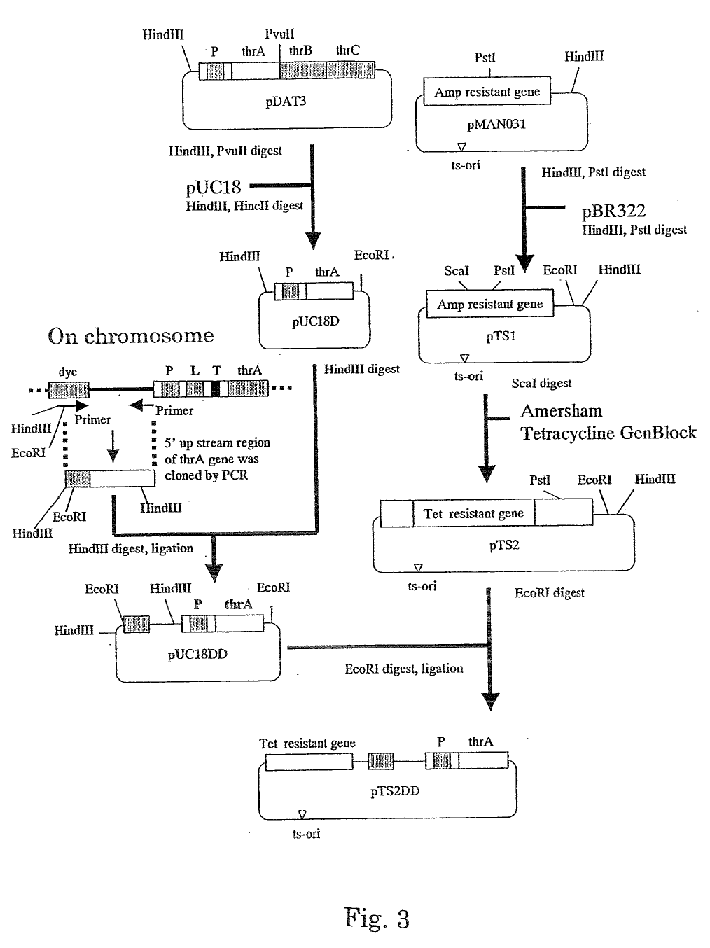

Then, for carrying out homologous recombination, the 5′ upstream sequence to the promoter of the chromosomal threonine operon was cloned by PCR as shown in FIG. 3. Specifically, DNA having nucleotide numbers 4454 to 6127 in the sequence of GENBANK registration number AE000510 was cloned. As the 5′ primer, an oligonucleotide which corresponds to a region covering both the 4458th A and the 4469th C, and replacing the 4458th A with T, and the 4469th C with T for introduction of HindIII and EcoRI sites was used. As the 3′ primer, an oligonucleotide complementary to a region on the 3′ side of the HindIII site (nucleotide numbers 6122 to 6127) in the sequence of GENBANK registration number AE000510 was used. By using these primers, a fragment of the region upstream to the threonine operon promoter was obtained. This fragment was digested with HindIII and inserted into the HindIII site of pUC18D to construct a plasmid pUC18DD.

Then, a temperature-sensitive plasmid for introducing a mutation into a chromosome was constructed. pBR322 (purchased from Nippon Gene) was digested with HindIII and PstI and the obtained fragment was introduced between the HindIII site and PstI site of pMAN031 (Yasueda, H. et al., Appl. Microbiol. Biotechnol., 36, 211 (1991)) to construct a temperature sensitive pTS1. Then, plasmid pTS2 having the antibiotic resistance gene replaced was constructed. That is, tetracycline GenBlock (purchased from Amersham) was inserted into the ScaI site within the ampicillin resistance gene of pTS1 to construct a temperature-sensitive plasmid pTS2.

Then, a temperature-sensitive plasmid for introducing a threonine operon having an attenuation-released type of sequence into a chromosome was constructed as follows. pUC18DD was digested with EcoRI, and the obtained fragment having a sequence encompassing the upstream and downstream to the attenuation region of the threonine operon was introduced into the EcoRI site of pTS2 to construct a plasmid pTS2DD for homologous recombination.

<3> Construction of a Strain Having a Threonine Operon Having an Attenuation-Released Type of Sequence on Chromosome

The temperature-sensitive plasmid pTS2DD was introduced into the W13 strain, namely, thrC-introduced TDH6 strain. The W13 strain was transformed with the temperature-sensitive plasmid pTS2DD, and colonies were selected at 30° C. on an LB+tetracycline plate. The selected clone was cultured overnight at 30° C., and the culture broth was diluted 103 times and inoculated on an LB+tetracycline plate to select colonies at 42° C. The selected clone was plated on an LB+tetracycline plate, cultured at 30° C., then transferred into a liquid medium and cultured at 42° C. for 4 to 5 hours with shaking. The culture broth was suitably diluted and inoculated on an LB plate. Several hundred colonies among the obtained colonies were selected and inoculated on an LB plate as well as an LB+tetracycline plate, and tetracycline-sensitive strains were selected. Colony PCR was performed for several of the tetracycline-sensitive strains to confirm whether the threonine operon having an attenuation-released type of sequence had been introduced. In this way, W13112 strain was constructed, which is a strain obtained by introducing thrC and attenuation-released type of threonine operon into TDH6. In the above operation, W1325 strain was also obtained having a wild-type attenuation region on a chromosome, except that thrC was introduced.

<4> Evaluation of L-Threonine Production by the Strain Introduced with a Threonine Operon Having an Attenuation-Released Type of Sequence on Chromosome

The W13112 strain lacking the leader sequence and attenuator in the attenuation region of the chromosomal threonine operon, and the control W1325 strain having a threonine operon with a wild-type attenuation region were respectively cultured by the method described in Example <2> of 1. The concentration of produced L-threonine was measured by the method described in Example <2> of 2. For each transformant, the amount of produced L-threonine in the presence of isoleucine is indicated as relative with respect to the amount of produced L-threonine in the absence of isoleucine, which was taken as 100. The results are shown in Table 4.

TABLE 4

| | |
|---|---|
| Added | Produced threonine |

| Strain | isoleucine (mg/L) | g/L | Relative value |
|---|---|---|---|
| W1325 | 0 | 2.9 | 100 |
| | 250 | 1.0 | 34 |
| W13112 | 0 | 5.6 | 100 |
| | 250 | 5.3 | 96 |

In the W13112 strain in which a chromosomal threonine operon has an attenuation-released type of sequence, the amount of accumulated L-threonine in the presence of isoleucine was high compared with the control strain, and thus it was demonstrated that L-threonine production was hardly affected by isoleucine added to the medium in the strain having a chromosomal threonine operon with an attenuation-released type of sequence.

### Example 4

### Construction and Evaluation of Strain in Which a Threonine Operon Having an Attenuation-Released Type of Sequence is Introduced on a Chromosome and which also Harbors a Plasmid Containing a Wild-Type of Threonine Operon

As shown in Example 3, in the strain in which the chromosomal threonine operon was replaced by that with an attenuation-released type of sequence, the amount of accumulated L-threonine was not decreased even in the presence of high concentrations of isoleucine. Then, the plasmid pVIC40 containing a threonine operon having a wild-type attenuation region was introduced into W13112 strain in order to confirm the effect of removal of the attenuation region from a chromosomal threonine operon.

W13112 was transformed with pVIC40, and transformants were selected for streptomycin-resistance. A transformant selected as a pVIC40-amplified strain was designated W13112/pVIC40, and plasmids were extracted. It was confirmed that the objective plasmid was amplified in the strain.

According to the method described in Example <2> of 1, L-threonine-producing ability of the W13112/pVIC40 strain was measured and compared with that of the control TDH6/pVIC40 strain containing the wild-type chromosomal threonine operon. For each transformant, the amount of produced L-threonine is represented as relative with respect to the amount of produced L-threonine in the absence of isoleucine, which is taken as 100. The results are shown in Table 5.

TABLE 5

| | Added | Produced |
|---|---|---|
| | isoleucine | L-threonine as |
| Strain | (mg/L) | relative value |
| TDH6/pVIC40 | 0 | 100 |
| | 250 | 60 |
| W13112/pVIC40 | 0 | 100 |
| | 250 | 77 |

In the TDH6/pVIC40 strain having a wild-type attenuation region of chromosomal threonine operon, the amount of produced threonine was markedly reduced with the addition of isoleucine. Conversely, in the W13112/pVIC40 strain in which the chromosomal threonine operon was of the attenuation-released type, the reduction in the amount of produced threonine in the presence of isoleucine was less significant as compared with the TDH6/pVIC40 strain.

### Example 5

### Measurement of L-Isoleucine-Producing Ability of a Strain Introduced with a Threonine Operon Having an Attenuation-Released Type of Sequence on the Chromosome

<1> Establishment of a L-Isoleucine-Producing Strain from the W13112/pVIC40 Strain and Evaluation Thereof.

L-isoleucine is produced via L-threonine as a precursor, and thus, an L-isoleucine-producing strain can be obtained by enhancing activities of L-isoleucine-biosynthetic enzymes in an L-threonine-producing bacterium (Japanese Patent Laid-open Nos. 09-121872 and 2002-051787). Therefore, in order to enhance activities of L-isoleucine-biosynthetic enzymes, the plasmid pMWD5 for amplifying genes for L-isoleucine-biosynthetic enzymes was introduced into the TDH6/pVIC40 strain and W13112/pVIC40 strain used in Example 5, respectively. Plasmid pMWD5 contains an isoleucine operon in which the region required for attenuation of the isoleucine operon itself is deleted (Japanese Patent Laid-open No. 09-121872). The plasmid pMWD5 was introduced into the each of TDH6/pVIC40 and W13112/pVIC40 by transformation as described in Example 5, and transformants were selected for ampicillin-resistance. The TDH6/pVIC40 strain having pMWD5 was designated TDH6/pVIC40 pMWD5, and the W13112/pVIC40 strain having pMWD5 was designated W31112/pVIC40 pMWD5.

<2> Evaluation of L-Isoleucine-Producing Ability of the Strain Introduced with a Threonine Operon Having an Attenuation-Released Type of Sequence on a Chromosome

Plasmids were extracted from the TDH6/pVIC40 pMWD5 strain and W31112/pVIC40 pMWD5 strain and it was confirmed that the objective plasmids were amplified in each strain.

Cells of the TDH6/pVIC40 pMWD5 strain and W31112/pVIC40 pMWD5 strain pre-cultured in the LB medium containing 20 μg/ml of streptomycin were respectively cultured in an L-isoleucine-production medium containing 40 g of glucose, 16 g of ammonium sulfate, 1 g of monopotassium phosphate, 0.01 g of ferrous sulfate heptahydrate, 0.01 g of manganese chloride tetrahydrate, 2 g of yeast extract, 1 g of magnesium sulfate heptahydrate and 30 g of calcium carbonate per 1 L of pure water (adjusted to pH 7.0 with KOH) at 37° C. for 22 to 27 hours with shaking at about 115 rpm.

After completion of the culture, the amount of L-threonine which had accumulated in each culture broth was analyzed for appropriately diluted culture broth by liquid chromatography. The results are shown in Table 6.

The yield of L-isoleucine obtained with the W13112/pVIC40 pMWD5 strain, which was a strain introduced with a threonine operon having an attenuation-released type of sequence on a chromosome, was improved as compared with the control TDH6/pVIC40 pMWD5 strain, and thus it was demonstrated that the removal of the attenuation region from the threonine operon was also effective for L-isoleucine production.

TABLE 6

| Strain | Produced L-isoleucine (g/L) |
|---|---|
| TDH6/pVIC40 pMWD5 | 10.1 |
| W31112/pVIC40 pMWD5 | 11.3 |

### INDUSTRIAL APPLICABILITY

According to the present invention, the yield of L-threonine and/or L-isoleucine can be improved during fermentation using a bacterium belonging to the genus Escherichia. In addition, the present invention provides a method for breeding of a novel L-threonine and/or L-isoleucine-producing bacterium.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, is incorporated by reference herein in its entirety.

1. An Escherichia bacterium which is able to produce L-threonine or L-isoleucine, wherein expression of a threonine operon therein is directed by its native promoter, and wherein at least a leader sequence and an attenuator has been deleted from said threonine operon.

2. The bacterium according to claim 1 wherein said threonine operon is on a plasmid.

3. The bacterium according to claim 1, wherein said threonine operon is on a chromosome.

4. The bacterium according to claim 1 which has an ability to produce L-isoleucine, and wherein an activity of an L-isoleucine-biosynthetic enzyme is enhanced.

5. The bacterium according to claim 1, wherein said leader sequence and said attenuator comprise at least nucleotides 188 to 310 of SEQ ID No. 1.

6. The bacterium according to claim 1 wherein said leader sequence and said attenuator comprise at least nucleotides 168 to 310 of SEQ ID No. 1.

7. The bacterium according to claim 1 wherein said leader sequence and said attenuator comprise at least nucleotides 148 to 310 of SEQ ID No. 1.

8. An isolated Escherichia threonine operon comprising a native promoter and thrABC, wherein at least a leader sequence and an attenuator sequence have been deleted from said operon.

9. The threonine operon according to claim 8 comprising the nucleotide sequence of SEQ ID NO: 1, wherein at least the sequence of nucleotides 188 to 310 has been deleted therefrom.

10. The threonine operon according to claim 8 comprising the nucleotide sequence of SEQ ID NO: 1, wherein at least the sequence of nucleotides 168 to 310 has been deleted therefrom.

11. The threonine operon according to claim 8 comprising the nucleotide sequence shown in SEQ ID NO: 1, wherein at least the sequence of nucleotides 148 to 310 has been deleted therefrom.

12. A method for producing L-threonine or L-isoleucine comprising culturing the bacterium according to claim 1 in a medium and collecting the L-threonine or L-isoleucine from the medium.
